(19) United States
(12) Patent Application Publication
(10) Pub. No.: US 2017/0303981 A1
(43) Pub. Date: Oct. 26, 2017

(54) SELF-RETAINING SCREWDRIVER TIP AND MATING DRIVE FEATURE

(71) Applicant: Symmetry Medical Manufacturing, Inc., Warsaw, IN (US)

(72) Inventor: Reese Myers, Columbia City, IN (US)

(73) Assignee: Symmetry Medical Manufacturing, Inc., Warsaw, IN (US)

(21) Appl. No.: 15/134,992

(22) Filed: Apr. 21, 2016

(51) Int. Cl. A61B 17/88; A61B 17/80

(52) U.S. Cl. CPC A61B 17/888; A61B 17/8057

(57) ABSTRACT

A surgical self-retaining screwdriver for the temporary securing of a bone fastener, wherein the tip of the screwdriver includes at least one deflection member which provides a frictional fit when the fastener is coupled to the screwdriver. A bone fastener is also provided which includes an undercut that is capable of temporarily retaining the at least one deflection member of the screwdriver. The invention provides a reliable means for securely holding a fastener to a screwdriver during insertion or extraction, and for easy removal of the screwdriver from the fastener after insertion.

### BACKGROUND OF THE INVENTION

### 1. Field of the Invention

The present invention relates to surgical instruments and implants, and, more particularly, to screwdrivers and bone screws.

### 2. Description of the Related Art

In the field of orthopedic surgery, bone fasteners such as screws have been used for many years and in many applications. Bone screws are used, for example, in securing a plate to the surface of a bone, securing an external fixator through the skin and into a bone, securing suture anchors into bone, and securing intramedullary devices inside the bone by passing the screw through both sides of the bone and the implant. Screws can also be used as implants themselves: for example, they can be used to secure bone fragments together, reinforce weak areas in bone, and fuse two or more different bones together in a joint.

When using bone screws, it is very often necessary to pass the screwdriver/screw combination through soft tissues prior to the desired ending location. For example, in a procedure where a bone plate is to be applied to the proximal femur of a patient, an incision is made and the plate is put into place. Following placement of the plate, the screw is placed on the end of the screwdriver and passed through the incision to the desired location on the plate, where it is then screwed into place.

Depending upon the amount of soft tissue between the skin and the bone, as well as the length of the incision or the angle of the path the screw takes, various problems can be encountered by the surgeon, who typically loses sight of the screw after it passes through the skin. The most common problem is the screw coming off the end of the screwdriver while it is in the soft tissues but not at its intended location. This situation can be very detrimental to the surgical procedure, as much time and effort can be expended upon finding the screw through visual, tactical, and/or x-ray location. In addition, the surgeon can experience much frustration.

Another problem that can arise is the screwdriver tip disassociating from the head of the screw after is in the desired location but not completely seated. The same soft tissues that the screw had to pass through on the way to the desired location can in some instances push against the screwdriver, or the surgeon may have inadvertently pulled the screwdriver away from the screw. In any event, more time and effort must be needed to re-attach the screwdriver to the screw, again leading to surgeon frustration and dissatisfaction.

Still another problem can be encountered when removing a bone screw. There are instances where the screw needs to be removed, either during the same procedure in which it was implanted, or after a previous implantation procedure. In any case, it is often difficult to locate the head of the screw for insertion of the tip of the screwdriver. When the screw is located and engaged, it is typically rotated counterclockwise and removed from the patient. At times, however, the screw may be difficult to remove completely. This can be as a result of soft tissues pressing against the head of the screw while it is being rotated, other obstructions in the bone inhibiting removal, or soft tissues pulling the screw from the screwdriver during the withdrawal process. Again, time and effort must be expended to re-engage the screwdriver and screw or even to locate the screw in the tissues.

Previous ways to alleviate these problems have been addressed in various ways. For example, “self-retaining” or “captured” surgical screwdrivers have been developed to attempt to temporarily hold the screw to the driver. Various mechanical means have been used: mechanical expansion of the screwdriver tip once it is inside the screw, mismatched geometries between the screwdriver tip and the driving portion of the screw head, sleeves on the screwdriver that descend over the screw head, threaded rods in the screwdriver that mate with threads in the head of the screw, etc. These are not optimal solutions, as manufacturing of the screwdriver and/or screw can be difficult and/or expensive, it is always desired to have the fewest components necessary to accomplish the task, and the connection between screwdriver and screw can be so great that they are not easily separable.

Another way to address the problem is a “low technology” solution: the surgeon simply wraps a loop of suture around the head of the screw after it is placed on the screwdriver tip, and grasps the free ends of the suture to hold the screw in place. After the screw is in the desired location, the surgeon ungrasps one end of the suture and pull the entire suture free from around the head of the screw and the wound itself. This is also not an optimal solution, because the hold is often precarious and prone to failure.

Yet another way to address the problem is by the use of cannulated screws. A surgical wire, commonly known as a K-wire, is placed in the bone at the desired location and out through the wound. A cannulated bone screw, i.e. a bone screw with a lengthwise through-hole, is placed over the K-wire and to the desired location. A cannulated screwdriver over the K-wire is then necessary to drive the screw, after which the screwdriver and K-wire are removed. There are times, however, when cannulated screws and screwdrivers may not be desired: the strength or function of the screws may demand a solid core, the screws may be too small to be cannulated, or the screw and/or screwdriver may be weakened by cannulation, among other reasons. In addition, manufacturing cost for the screwdriver and screw are greater than their non-cannulated counterparts.

What is needed in the art is a surgical screwdriver that efficiently and predictably holds a bone screw in a secure manner until it is no longer required to do so by the surgeon. What is also needed in the art are bone screws which are adapted to optimally couple with surgical screwdrivers, without complicated manufacturing methods and subsequent cost.

### SUMMARY OF THE INVENTION

The present invention is directed to an improved surgical screwdriver incorporating features that enable it to temporarily yet securely retain a bone fastener such as a screw, yet be manufacturable by conventional means.

The present invention provides a surgical screwdriver with a tip especially designed to temporarily secure a bone screw while it is being transported and/or driven. One or more deflection members are disposed on the tip and, upon insertion into a screw head, deflect under elastic deformation as a result of a portion of the tip having a larger circumferential dimension than the instrument-receiving portion of the screw. After the screwdriver tip is seated in the screw, the deflection member's urging against the inside of the screw provides a friction between the two that is adequate for the purpose of temporary retention.

The present invention also provides a bone fastener, particularly a bone screw, which may be attached to a surgical screwdriver with a tip especially designed to temporarily secure it while it is being transported and/or driven. In addition to the instrument-receiving portion of the screw having a geometry which substantially is the same as the driving end of a screwdriver, a cutout is also included for temporary securing the inventive surgical screwdriver, as well as any screwdriver with a complementary feature on its driving end.

An advantage of the present invention is that as a result of the simplicity of the design, the screwdriver tip may be easier to clean than currently-available screw-retaining features on screwdrivers such as circular retaining rings, clips, sleeves, or threaded rods.

Another advantage of the present invention is compared to screwdrivers that use silicone rings or other non-integral components, it may provide a more secure fixation of the screw.

Yet another advantage of the present invention is that as a result of the simplicity of the design, the screwdriver may be less costly to manufacture than screwdrivers with screw-retaining features that have tightly-toleranced features relative to the mating screw features.

Another advantage of the present invention is that as a result of the simplicity of the design, the screwdriver may be less expensive than screwdrivers with screw-retaining means provided by complicated multi-member mechanisms.

Still another advantage of the present invention is that as a result of not adding other components and having a profile not much larger than the shaft, the screwdriver may provide a less-obstructed view of the screw insertion site than screwdrivers with screw-retaining clamps attached to the driver shaft.

Yet another advantage of the present invention is the fastener head is essentially undisturbed with the exception of an easily-machined undercut.

### BRIEF DESCRIPTION OF THE DRAWINGS

The above-mentioned and other features and advantages of this invention, and the manner of attaining them, will become more apparent and the invention will be better understood by reference to the following description of an embodiment of the invention taken in conjunction with the accompanying drawings, wherein:

FIG. 1 is a perspective view of an embodiment of a surgical screwdriver of the present invention;

FIG. 2 is a side view of the distal end of the surgical screwdriver of FIG. 1;

FIG. 3 is a sectioned perspective view of an embodiment of a bone fastener of the present invention;

FIG. 4 is a perspective view of the surgical screwdriver of FIG. 1 with the bone fastener of FIG. 3 prior to coupling; and

FIG. 5 is a sectioned perspective view of the surgical screwdriver of FIG. 1 with the bone fastener of FIG. 3 after coupling.

Corresponding reference characters indicate corresponding parts throughout the several views. The exemplification set out herein illustrates an embodiment of the invention, in one form, and such exemplification is not to be construed as limiting the scope of the invention in any manner.

### DETAILED DESCRIPTION OF THE INVENTION

The terms “proximal” and “distal” are used principally throughout this specification for convenience; but it is to be understood that these terms are not intended to be limiting. Thus “proximal” in this specification refers to the feature of the apparatus closest to the operator during use, and “distal” refers to the end of the apparatus farthest from the operator during use.

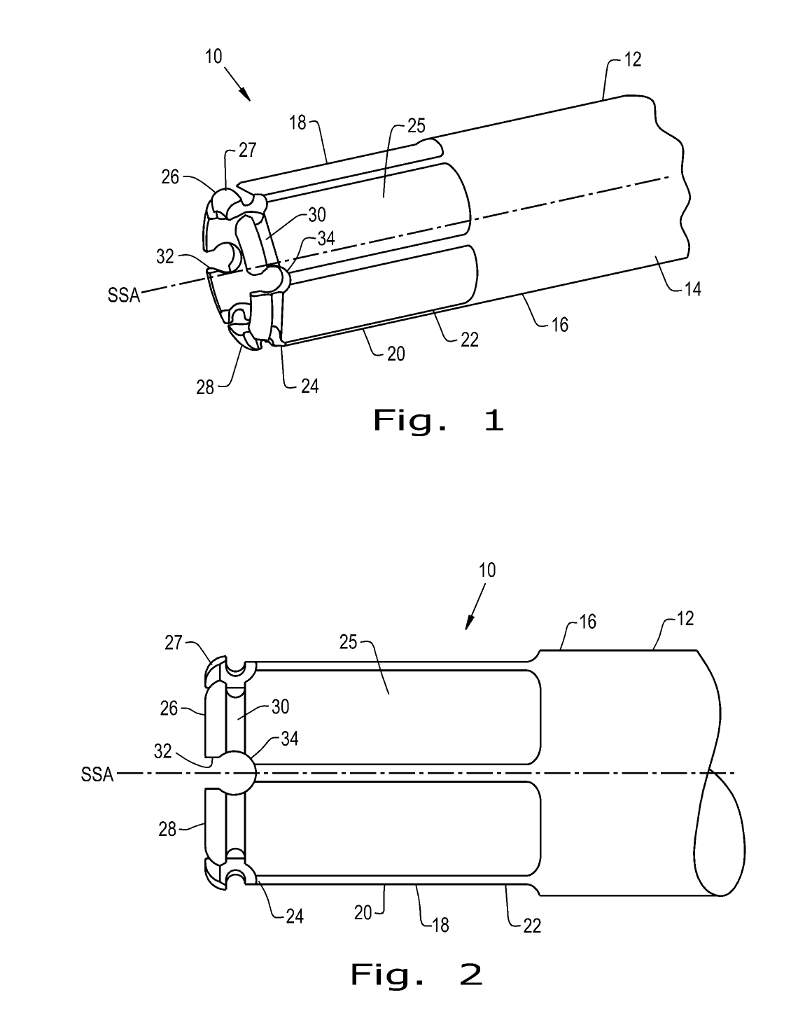

Referring now to FIGS. 1-2, there is shown a surgical screwdriver 10 according to the present invention. Surgical screwdriver 10 can be made of any metal, composite, or plastic suitable for use with surgical instruments. The surgical screwdriver 10 includes a shaft 12 with proximal end 14, distal end 16, and longitudinal axis SSA. Proximal end 14 may be attached to a separate handle, formed into its own handle, or used with a power tool, none of which is shown. Shaft 12 may be solid or cannulated (includes a through-hole coincident with the longitudinal axis SSA).

Distal end 16 of shaft 12 includes a fastener-engaging tip 18. Fastener-engaging tip 18 can be any length necessary for secure engagement with a fastening device, and includes a driving portion 20 with proximal end 22, distal end 24, and at least one driving face 25. Driving portion 20, in the exemplary embodiment, has a cross-sectional geometry defining a hexagon. However, the cross-sectional geometry is not limited to hexagonal and can be any geometry suitable for use as a screwdriver; including but not limited to octagonal, square, star-shaped, etc. Therefore, in the exemplary embodiment, driving portion 20 has six driving faces 25: one for each side of the cross-sectional geometry.

Distal end 24 of driving portion 20 includes retaining portion 26. Retaining portion 26 includes at least one deflection member 28 attached to the at least one driving face 25. In the exemplary embodiment, retaining portion 20 includes six deflection members 28, one for each driving face 25. However, as the cross-sectional geometry of the driving portion 20 is not limited to hexagonal and can be any geometry suitable for use as a screwdriver; including but not limited to octagonal, square, star-shaped, etc.; there can be a corresponding number of driving faces 25 with deflection members 28. In other embodiments not shown with a plurality of driving faces 25, there may only be one deflection member 28 attached to one of the plurality of driving faces 25, the other driving faces 25 having no deflection members 28 attached to them. Any combination of driving faces 25 and attached deflection members 28 is therefore possible. Retaining portion 26 can be fixedly or removably attached to fastener-engaging tip 18.

As best seen in FIG. 2, the at least one deflection member 28 includes protrusion 27 and undercut 30. Protrusion 27 can have any configuration; in the exemplary embodiment it is radiused for ease of insertion and temporary coupling with the inventive bone fastener as described below. Undercut 30, when viewed in a cross-section parallel to longitudinal axis SSA, defines a radius.

By virtue of its design, especially considering undercut 30, the at least one deflection member 28 is capable of elastic deformation when subjected to external loads; that is, it can be bent at its attachment to driving face 25 a limited amount and return to its original position. The amount of elastic deformation possible is a function of the material properties, the design, and the forces applied.

Advantageously, the purpose for which being detailed further in this specification, a cross-sectional diameter circumscribing the surgical screwdriver 10 which includes the at least one deflection member 28 is larger (has a greater diameter) than a cross-sectional diameter circumscribing the surgical screwdriver 10 which includes the driving portion 20 but does not include the at least one deflection member 28.

In the exemplary embodiment shown in FIGS. 1-2, there are a plurality of driving faces 25 and deflection members 28 on surgical screwdriver 10. In this embodiment, or in any embodiment with a plurality of driving faces 25 and deflection members 28, an opening 32 is included between any two deflection members 28 which are located next to each other as shown. At the base of each opening 32—that is, at the distal end of driving portion 20—is a radiused notch 34. The purpose of radiused notch 34 is to provide stress relief to surgical screwdriver 10 upon plastic deformation of the at least one deflection member 28, and can be of any suitable geometry to serve that purpose.

It is to be understood that in the event of an embodiment with a single deflection member 28 and the absence of an opening 32 (not shown), a radiused notch 34 is included on each side of the deflection member 28 in the location described above.

It is also to be understood that retaining portion 26 can be fixedly or removably attached to fastener-engaging tip 18.

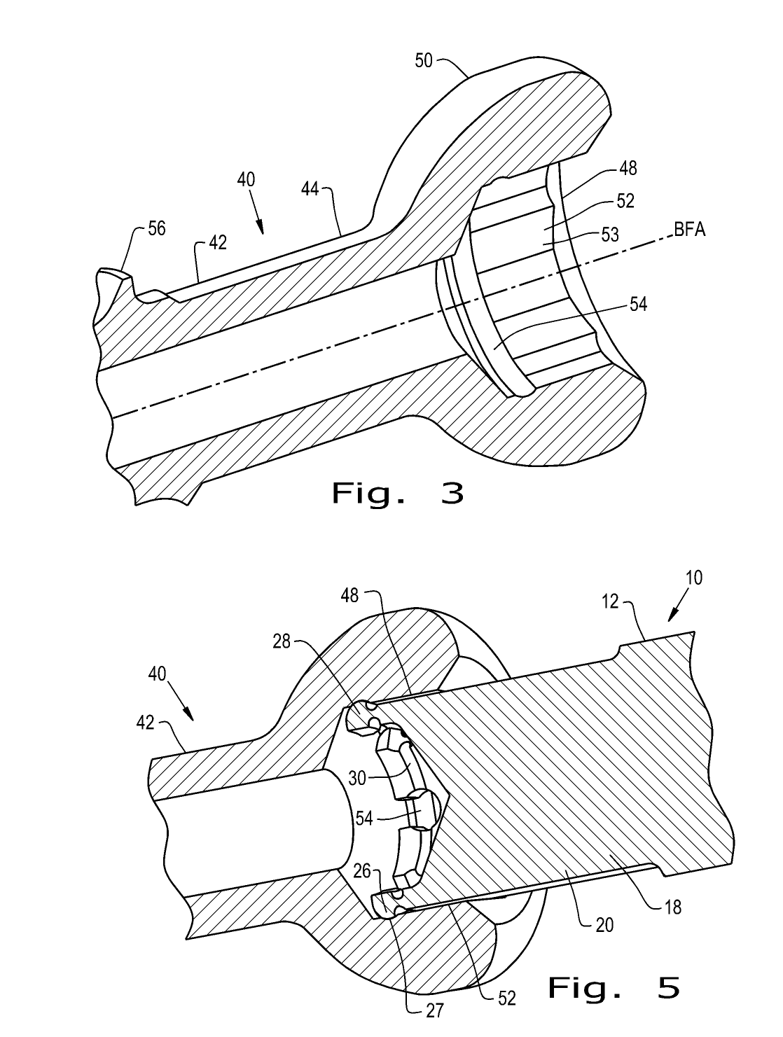

Now referring to FIG. 3, there is shown an embodiment of a bone fastener 40 according to the present invention. Bone fastener 40 can be made of any metal or biodegradable material suitable for use with surgical implants. The bone fastener 40 includes shaft 42 with proximal end 44, distal end 46, and longitudinal axis BFA. Proximal end 44 includes head 50, which includes instrument-receiving portion 48. Shaft 42 may be solid or cannulated (includes a through-hole coincident with the longitudinal axis BFA).

Bone- or plate-engaging threads 56 may be disposed completely or partially on the shaft 42 of bone fastener 40; therefore, they may also be disposed completely or partially on the head 50 (not shown). The bone- or plate-engaging threads 56 may be of any type known in the art.

Instrument-receiving portion 48 includes female opening 52, which includes driven faces 53 and undercut 54. In the exemplary embodiment show, there are twelve driven faces 53, which corresponds to the driving tip of a screwdriver with six or twelve driving faces. The cross-section of the head 50 through the driven faces 53, then, defines a dodecagon. However, the cross-sectional geometry is not limited to a dodecagon and can be any geometry suitable for use; including but not limited to octagonal, hexagonal, square, star-shaped, etc.

Undercut 54, when viewed in a cross-section parallel to longitudinal axis BFA, defines a radius in the exemplary embodiment. However, undercut 54 can have any configuration; in the exemplary embodiment it is radiused for ease of insertion and temporary coupling with the inventive surgical screwdriver as described below. Undercut 54 may be disposed completely or partially around the inside of the female opening 52; and may be located distal to, proximal to, or anywhere along driven faces 53.

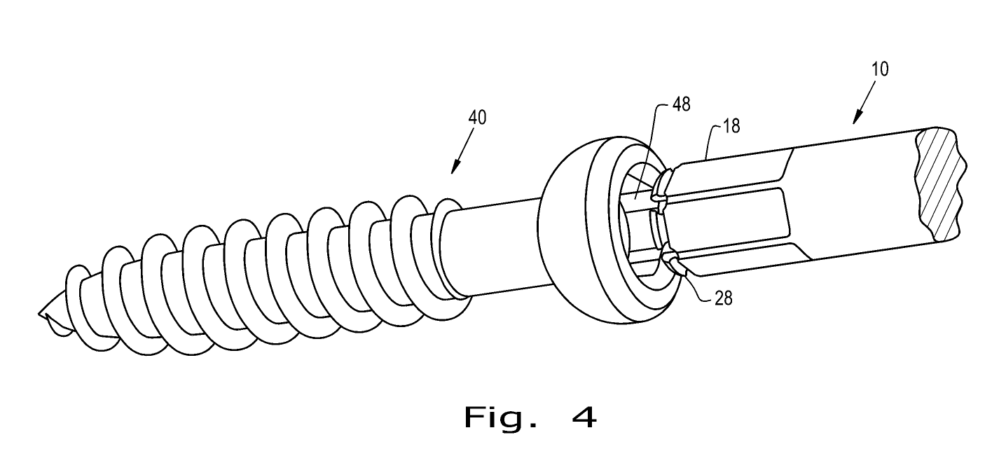

Now referring to FIGS. 4 and 5, the operation of inventive surgical screwdriver 10 and the inventive bone fastener 40 is described. Prior to transporting the screw to its desired location, or prior to removing the screw from its current location, the fastener-engaging tip 18 of surgical screwdriver 10 is inserted into the instrument-receiving portion 48 of bone fastener 40.

In the embodiment shown, a cross-sectional diameter circumscribing the surgical screwdriver 10 which includes the at least one deflection member 28 is larger (has a greater diameter) than a cross-sectional diameter circumscribing the instrument-receiving portion 48 of the bone fastener 40 with accompanying driven faces 53. Therefore, as the fastener-engaging tip 18 is inserted into instrument-receiving portion 48, the at least one deflection member 28 is forced inward toward the longitudinal axes SSA and BFA under conditions of elastic deformation; in other words, protrusion 27 on at least one deflection member 28 intimately contacts a surface of the at least one of the driven faces 53.

In the embodiment shown, when the fastener-engaging tip 18 of surgical screwdriver 10 is completely inserted into the instrument-receiving portion 48 of bone fastener 40, the protrusion 27 of the at least one deflection member 28 is free to return to or near to its pre-insertion position by virtue of its ability to move into and within undercut 54, since protrusion 27 is no longer contacting one of the driven faces 53. As described above, the undercut 54 and protrusion 27 may be complementary (have substantially the same geometries), or may differ from one another in geometry.

The bone fastener 40 may then be transported to the desired site and screwed into place, after which surgical screwdriver 10 can be disassociated with bone fastener 40 by simply pulling back from it. Alternatively, in the event of screw removal, the bone fastener 40 may then be unscrewed and transported away from its original location to outside the wound, where it can then be disassociated from surgical screwdriver 10 by pulling them in opposite directions.

While self-retaining screwdrivers and bone fasteners have been described with respect to at least one embodiment, the present invention can be further modified within the spirit and scope of this disclosure. This application is therefore intended to cover any variations, uses, or adaptations of the invention using its general principles. Further, this application is intended to cover such departures from the present disclosure as come within known or customary practice in the art to which this invention pertains and which fall within the limits of the appended claims.

What is claimed is:

1. A surgical screwdriver comprising:
a shaft with a proximal end, a distal end, and a longitudinal axis; and
a fastener-engaging tip attached to the distal end of the shaft; the fastener-engaging tip including:
a driving portion with a proximal end, a distal end, at least one driving face, and a cross-sectional geometry; and
a retaining portion attached to the distal end of the driving portion, the retaining portion including at least one deflection member, the at least one deflection member including a protrusion, the protrusion of the at least one deflection member capable of movement toward the longitudinal axis, the retaining portion having a cross-sectional diameter which is larger than a cross-sectional diameter of the driving portion.

2. The surgical screwdriver of claim 1, wherein the cross-sectional geometry of the driving portion defines a hexagon.

4. The surgical screwdriver of claim 1, wherein the at least one deflection member, includes an undercut which, when viewed in a cross-section parallel to the longitudinal axis, defines a radius.

5. The surgical screwdriver of claim 1, wherein the at least one deflection member is a plurality of deflection members.

6. The surgical screwdriver of claim 1, wherein the at least one deflection member includes at least one radiused notch at the distal end of the driving portion.

7. The surgical screwdriver of claim 5, wherein the plurality of deflection members includes an opening between any two of the plurality of deflection members, the opening including at least one radiused notch at the distal end of the driving portion.

8. The surgical screwdriver of claim 1, wherein the retaining portion is removably attached.

9. A bone fastener comprising:
a shaft with a proximal end, a distal end, and a longitudinal axis; and
a head with an instrument-receiving portion attached to the proximal end of the shaft; the instrument-receiving portion including:
a female opening in the head with a cross-sectional geometry and an inner surface for receivable engagement of a fastener-engaging tip of a surgical screwdriver; and
an undercut extending at least partially circumferentially around the inner surface of the instrument-receiving portion.

10. The bone fastener of claim 9, wherein at least one of bone- and plate-engaging threads are at least one of completely or partially disposed along the shaft.

11. The bone fastener of claim 9, wherein the head includes at least one of bone- and plate-engaging threads.

12. The bone fastener of claim 9, wherein the cross-sectional geometry of the female opening defines a hexagon.

13. The bone fastener of claim 9, wherein the undercut, when viewed in a cross-section parallel to the longitudinal axis, defines a radius.

14. A method of temporarily securing a bone fastener to a surgical screwdriver, the method comprising:
providing a surgical screwdriver including:
a shaft with a proximal end, a distal end, and a longitudinal axis; and
a fastener-engaging tip attached to the distal end of the shaft; the fastener-engaging tip including:
a driving portion with a proximal end, a distal end, at least one driving face, and a cross-sectional geometry; and
a retaining portion attached to the distal end of the driving portion, the retaining portion including at least one deflection member, the at least one deflection member including a protrusion, the protrusion of the at least one deflection member capable of movement toward the longitudinal axis, the retaining portion having a cross-sectional diameter which is larger than a cross-sectional diameter of the driving portion;
providing a bone fastener including:
a shaft with a proximal end, a distal end, and a longitudinal axis; and
a head with an instrument-receiving portion attached to the proximal end of the shaft, the instrument-receiving portion including a female opening with a cross-sectional geometry and an inner surface, the inner surface including an undercut extending at least partially circumferentially for the receivable engagement of the fastener-engaging tip of the surgical screwdriver; and
placing the bone fastener onto the surgical screwdriver where it is temporarily held as a result of the protrusion of the at least one deflection member residing within the undercut.

15. The method of claim 14, wherein the cross-sectional geometry of the driving portion defines a hexagon.

16. The method of claim 14, wherein the at least one deflection member, includes an undercut which, when viewed in a cross-section parallel to the longitudinal axis, defines a radius.

17. The method of claim 14, wherein the at least one deflection member is a plurality of deflection members.

18. The method of claim 14, wherein the at least one deflection member includes at least one radiused notch at the distal end of the driving portion.

19. The method of claim 17, wherein the plurality of deflection members includes an opening between any two of the plurality of deflection members, the opening including at least one radiused notch at the distal end of the driving portion.

20. The method of claim 14, wherein the retaining portion is removably attached.
